(19) United States
(12) Patent Application Publication
(10) Pub. No.: US 2012/0226174 A1
(43) Pub. Date: Sep. 6, 2012

(54) PULSE WAVE VELOCITY MEASUREMENT DEVICE AND PULSE WAVE VELOCITY MEASUREMENT PROGRAM

(76) Inventors: Yutaka Ikeda, Osaka-shi (JP); Atsushi Hori, Osaka-shi (JP)

(21) Appl. No.: 13/508,933

(22) PCT Filed: Nov. 1, 2010
(86) PCT No.: PCT/JP2010/069408

§ 371 Date: May 9, 2012

(30) Foreign Application Priority Data
Nov. 10, 2009 (JP) 2009-257217

(51) Int. Cl. A61B 5/024

(52) U.S. Cl. 600/500

(57) ABSTRACT

In a pulse wave velocity measurement device (10), reference times (T1, T2) for an ejection wave component (S1) and a reflected component (S2) of a pulse wave S are detected by a reference time detection unit (2), and amplitudes (W1, W2) of the pulse wave (S) that correspond to the reference times (T1, T2) for the ejection wave component (S1) and the reflected wave component (S2) are detected by a pulse wave amplitude detection unit (3). A pulse wave velocity detection unit (5) finds a velocity (PWV1) of the pulse wave (S) on basis of the reference times (T1, T2) for the ejection wave component (S1) and the reflected wave component (S2) and the amplitudes (W1, W2) of the pulse wave (S) that correspond to the reference times (T1, T2) for the ejection wave component (S1) and the reflected wave component (S2). Thus the pulse wave velocity can be measured with high accuracy in consideration of a difference between the pulse wave velocities of the ejection wave and the reflected wave which difference is caused by a difference between the amplitude of the ejection wave component (S1) and the amplitude of the reflected wave component (S2).

### TECHNICAL FIELD

The present invention relates to a pulse wave velocity measurement device and a pulse wave velocity measurement program for measuring pulse waves of a living body and thereby calculating velocities at which the pulse waves are propagated.

### BACKGROUND ART

It has conventionally been known that pulse waves have various important information for grasp on a state of a circulatory system of a living body. In particular, a velocity and time for propagation of a pulse wave between two sites in a living body are living-body indices that have drawn attention from the medical work front because a possibility has been pointed out that a state of arteriosclerosis may be grasped by the indices, and are called Pulse Wave Velocity (PWV) and Pulse Transit Time (PTT), respectively, or the like.

A plurality of measurement methods according to measurement sites have been proposed for the pulse wave velocity, and the pulse wave velocity between a carotid artery and a femoral artery, for instance, is called cfPWV (carotid-femoral PWV) and is used as a gold standard of the pulse wave velocity (PWV).

In general, two measurement points are required in order to find the pulse wave velocity (PWV).

It is known, however, that a pulse wave measured at any site on a living body is a resultant wave of an ejection wave from the heart and reflected waves reflected from various sites in the living body. There is a possibility that separation into the ejection wave and the reflected waves may make it possible to find the pulse wave velocity or the pulse transit time even it only one measurement point for pulse waves exists.

In Non-Patent Literature 1, therefore, a technique for separating the ejection wave and the reflected waves is disclosed. In Patent Literature 1 and Patent Literature 2, there are disclosed techniques for finding the pulse wave velocity or the pulse transit time by separating the ejection wave and the reflected waves even in presence of only one measurement point for pulse waves.

In general, reflection of pulse waves occurs at various sites in a living body. The reflection of pulse waves is caused mainly by impedance mismatch in blood vessels and thus occurs at sites having blood vessel bifurcation, variation in elastic force in blood vessel or the like, for instance.

As described in Patent Literature 1, the separation into the ejection wave and the reflected waves is satisfactorily attained for pulse waves measured at various sites in a living body, on an assumption that a staple reflection point is in vicinity of iliac arteries or an abdominal aorta.

Disclosed in Patent Literature 2 is a technique for calculating the pulse wave velocity or the pulse transit time and finding a blood pressure from pulse waves obtained at one measurement point on basis of a correlation between the pulse wave velocity or the pulse transit time and the blood pressure. Disclosed in Non-Patent Literature 2 is a technique for calculating the pulse wave velocity or the pulse transit time and finding a blood pressure from pulse waves obtained at two measurement points.

It is disclosed in both Patent Literatures 1 and 2 described above that the pulse wave velocity or the pulse transit time can be found on basis of pulse waves at the one measurement point, and this is based on a premise that the pulse wave velocities of the ejection wave and the reflected waves are equal. According to the premise, (1) one pulse wave is separated into an ejection wave and a reflected wave and a time difference between both the waves is found, and (2) a difference between pulse wave propagation distances of the ejection wave and the reflected wave is found. The pulse wave velocity or the pulse transit time is calculated therefrom.

The pulse wave velocities of the ejection wave and the reflected wave, however, do not actually coincide with each other. That is because amplitudes of the ejection wave and the reflected wave differ. As noted in Patent Literature 2 described above, the pulse wave velocity is correlated with the blood pressure, which relates to the amplitude of the pulse wave. That is, the pulse wave velocity relates to the amplitude of the pulse wave.

As described above, a waveform of a pulse wave can satisfactorily be explained on assumption that the reflection point of the reflected wave is in vicinity of the abdominal aorta. Though the reflection of pulse waves is caused by the impedance mismatch in aortas and the abdominal aorta, degrees of the mismatch differ among individuals and according to physical conditions and conditions of blood vessels in one individual. As a result, the amplitude of the reflected wave differs from that of the traveling wave, and degrees of the difference differ among individuals. This results in a difference in the pulse wave velocity between the ejection wave and the reflected wave.

### CITATION LIST

### Patent Literature

Patent Literature 1: JP 2003-010139 A

Patent Literature 2: JP 2007-007075 A

Non-Patent Literature 1: Takazawa K at al. “Underestimation of vasodilator effects of nitroglycerin by upper limb blood pressure”, Hypertension 1995; 26:520-3

Non-Patent Literature 2: McCombie, Devin “Development of a wearable blood pressure monitor using adaptive calibration of peripheral pulse transit time measurements”, Ph.D. Thesis, Massachusetts Institute of Technology, Dept. of Mechanical Engineering, 2008.

### SUMMARY OF INVENTION

### Technical Problem

Therefore, an object of the invention is to provide a pulse wave velocity measurement device and a pulse wave velocity measurement program by which a pulse wave velocity can more accurately be found on basis of pulse waves at one measurement site.

### Solution to Problem

In order to solve the problem, a pulse wave velocity measurement device according to the present invention comprises:

a pulse wave detection unit for detecting a pulse wave at one site in a living body,

a reference time detection unit for detecting reference time for identification of an ejection wave component included in the pulse wave at the one site that is detected by the pulse wave detection unit and reference time for identification of a reflected wave component included in the pulse wave,

a pulse wave amplitude detection unit for detecting an amplitude of the pulse wave that corresponds to the reference time for the ejection wave component detected by the reference time detection unit and detecting an amplitude of the pulse wave that corresponds to the reference time for the reflected wave component detected by the reference time detection unit,

a pulse wave velocity detection unit for finding a velocity of the pulse wave at which the pulse wave propagates, on basis of the reference time for the ejection wave component and the reference time for the reflected wave component that have been detected by the reference time detection unit and the amplitude of the pulse wave that corresponds to the reference time for the ejection wave component and the amplitude of the pulse wave that corresponds to the reference time for the reflected wave component which amplitudes have been detected by the pulse wave amplitude detection unit.

The pulse wave velocity measurement device of the invention detects the reference times for the ejection wave component and for the reflected wave component of the pulse wave by the reference time detection unit and detects the amplitudes of the pulse wave that correspond to the reference times for the ejection wave component and for the reflected wave component by the pulse wave amplitude detection unit. The pulse wave velocity detection unit finds the velocity of the pulse wave on basis of the reference times for the ejection wave component and for the reflected wave component, and the amplitudes of the pulse wave that correspond to the reference times for the ejection wave component and for the reflected wave component. Thus the pulse wave velocity can be measured with high accuracy in consideration of a difference between the pulse wave velocities of the ejection wave and the reflected wave which difference is caused by a difference between the amplitude of the ejection wave component and the amplitude of the reflected wave component.

In a pulse wave velocity measurement device according to one embodiment, the pulse wave amplitude detection unit includes an ejection wave component elimination unit for finding an ejection wave component included in the amplitude of the pulse wave that corresponds to the reference time for the reflected wave component, eliminating the ejection wave component from the amplitude of the pulse wave that corresponds to the reference time for the reflected wave component, and thereby finding an amplitude of the reflected wave component that corresponds to the reference time for the reflected wave component, and

the pulse wave velocity detection unit finds the velocity of the pulse wave on basis of the reference time for the ejection wave component and the reference time for the reflected wave component that have been detected by the reference time detection unit, the amplitude of the pulse wave that corresponds to the reference time for the ejection wave component that has been detected by the pulse wave amplitude detection unit, and the amplitude of the reflected wave component that has been found by the ejection wave component elimination unit.

According to the embodiment, the ejection wave component elimination unit eliminates the ejection wave component from the amplitude of the pulse wave that corresponds to the reference time for the reflected wave component and thereby finds the amplitude of the reflected wave component that corresponds to the reference time for the reflected wave component, so that the amplitude of the reflected wave component that corresponds to the reference time for the reflected wave component can accurately be found. Therefore, the velocity of the pulse wave can accurately be found by the pulse wave velocity detection unit.

In a pulse wave velocity measurement device according to one embodiment, the pulse wave velocity detection unit finds the velocity of the pulse wave on basis of a time difference between the reference time for the ejection wave component and the reference time for the reflected wave component that have been detected by the reference time detection unit, and an amplitude difference between the amplitude of the pulse wave that corresponds to the reference time for the ejection wave component and that has been detected by the pulse wave amplitude detection unit and the amplitude of the reflected wave component that has been found by the ejection wave component elimination unit.

According to the embodiment, the velocity of the pulse wave can accurately be found on basis of the time difference between the reference time for the ejection wave component and the reference time for the reflected wave component that have been detected by the reference time detection unit and on basis of the amplitude difference between the amplitude of the pulse wave that corresponds to the reference time for the ejection wave component that is detected by the pulse wave amplitude detection unit and the amplitude of the reflected wave component that has been found by the ejection wave component elimination unit.

A pulse wave velocity measurement program that makes a computer according to one embodiment, executes:

a reference time deriving function of finding reference time for identification of an ejection wave component included in a pulse wave at one site in a living body and reference time for identification of a reflected wave component included in the pulse wave,

a pulse wave amplitude deriving function of finding an amplitude of the pulse wave that corresponds to the reference time for the ejection wave component and finding an amplitude of the pulse wave that corresponds to the reference time for the reflected wave component, and

a pulse wave velocity deriving function of calculating a velocity of the pulse wave at which the pulse wave propagates, on basis of the reference time for the ejection wave component, the reference time for the reflected wave component, the amplitude of the pulse wave that corresponds to the reference time for the ejection wave component, and the amplitude of the pulse wave that corresponds to the reference time for the reflected wave component.

According to the pulse wave velocity measurement program of the embodiment, the computer is made to execute the reference time deriving function to find the reference times for the identification of the ejection wave component and the reflected component of the pulse wave, and the pulse wave amplitude deriving function to find the amplitudes of the pulse wave that correspond to the reference times for the identification of the ejection wave component and the reflected component. The velocity of the pulse wave is then calculated by the pulse wave velocity deriving function on basis of the reference times for the identification of the ejection wave component and the reflected component and the amplitudes of the pulse wave that correspond to the reference times for the identification of the ejection wave component and the reflected component. Thus the pulse wave velocity can be detected with higher accuracy in consideration of the difference between the pulse wave velocities of the ejection wave and the reflected wave which difference is caused by a difference between the amplitude of the ejection wave component and the amplitude of the reflected wave component.

In a pulse wave velocity measurement program according to one embodiment, the pulse wave amplitude deriving function includes an ejection wave component eliminating function of finding an ejection wave component included in the amplitude of the pulse wave that corresponds to the reference time for the reflected wave component, eliminating the ejection wave component included in the amplitude of the pulse wave from the amplitude of the pulse wave that corresponds to the reference time for the reflected wave component, and thereby finding an amplitude of the reflected wave component that corresponds to the reference time for the reflected wave component, and

the pulse wave velocity deriving function comprises calculating the velocity of the pulse wave on basis of the reference time for the ejection wave component, the reference time for the reflected wave component, the amplitude of the pulse wave that corresponds to the reference time for the ejection wave component, and the amplitude of the reflected wave component that has been found by the ejection wave component eliminating function.

According to the pulse wave velocity measurement program of the embodiment, the ejection wave component included in the amplitude of the pulse wave is eliminated by the ejection wave component eliminating function from the amplitude of the pulse wave that corresponds to the reference time for the reflected wave component, and the amplitude of the reflected wave component that corresponds to the reference time for the reflected wave component is thereby found. Thus the amplitude of the reflected wave component that corresponds to the reference time for the reflected wave component can more accurately be found. Therefore, the velocity of the pulse wave can more accurately be found by the pulse wave velocity deriving function.

In a pulse wave velocity measurement program according to one embodiment, the pulse wave velocity deriving function comprises finding the velocity of the pulse wave on basis of a time difference between the reference time for the ejection wave component and the reference time for the reflected wave component, and an amplitude difference between the amplitude of the pulse wave that corresponds to the reference time for the ejection wave component and the amplitude of the reflected wave component that has been found by the ejection wave component eliminating function.

According to the embodiment, the velocity of the pulse wave can accurately be found on basis of the time difference between the reference time for the ejection wave component and the reference time for the reflected wave component and the amplitude difference between the amplitude of the pulse wave that corresponds to the reference time for the ejection wave component and the amplitude of the reflected wave component that has been found by the ejection wave component eliminating function.

### Advantageous Effects of Invention

According to the pulse wave velocity measurement device of the invention, the pulse wave velocity is found on basis of the reference times for the identification of the ejection wave component and the reflected component and the amplitudes of the pulse wave that correspond to the reference times for the identification of the ejection wave component and the reflected component, and thus the pulse wave velocity can be detected with high accuracy in consideration of the difference between the pulse wave velocities of the ejection wave and the reflected wave which difference is caused by the difference between the amplitude of the ejection wave component and the amplitude of the reflected wave component.

### BRIEF DESCRIPTION OF DRAWINGS

FIG. 1 is a block diagram of a pulse wave velocity measurement device that is an embodiment of the invention;

FIG. 2A is a waveform chart showing a waveform of a pulse wave detected by a pulse wave detection unit in the embodiment;

FIG. 2B is a waveform chart showing a waveform of an acceleration wave of the pulse wave;

FIG. 2C is a waveform chart showing a waveform of a third derivative of the pulse wave detected by the pulse wave detection unit in the embodiment;

FIG. 2D is a waveform chart showing a waveform of a fourth derivative of the pulse wave;

FIG. 3 is a waveform chart showing a waveform of the pulse wave and amplitudes W1, W2 of the pulse wave at reference points Q1, Q2 of the pulse wave;

FIG. 4 is a waveform chart showing a waveform of the pulse wave, and an ejection wave component S1 and a reflected wave component S2 of the pulse wave; and

FIG. 5 is a schematic diagram that schematically shows a path d through which the ejection wave component S1 of the pulse wave S propagates in a human living body and a path h through which the reflected wave component S2 propagates in the human living body.

### DESCRIPTION OF EMBODIMENTS

Hereinbelow, the invention will be described in detail with reference to an embodiment shown in the drawings.

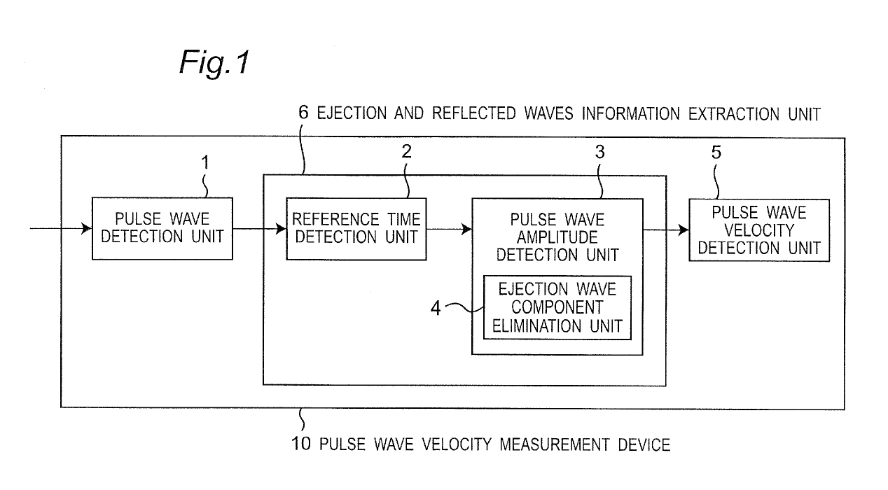

FIG. 1 is a block diagram showing a pulse wave velocity measurement device 10 that is an embodiment of the invention. The pulse wave velocity measurement device 10 includes a pulse wave detection unit 1 that detects pulse waves at one site in a living human body. The pulse wave detection unit 1 may be, for instance, a unit in which a degree of reflection or absorption of infrared light outputted from a light emitting device according to a quantity of blood in a blood vessel is measured by a light receiving device (photoplethysmography). Alternatively, the pulse wave detection unit 1 may be used a unit that detects pulse waves by pressure pulse wave method in which a change in pressure of blood in a blood vessel against the blood vessel is extracted as electric signals. Especially severe limitations do not exist on the site in a living body where pulse waves are detected by the pulse wave detection unit 1, whereas the site is desirably set at a site noninvasive and unconstrained, if possible, and is preferably set on a finger tip, a wrist, an earlobe or the like.

The pulse wave velocity measurement device 10 includes an ejection and reflected waves information extraction unit 6 for detecting reference times for identification of an ejection wave component and a reflected wave component that are included in the pulse wave detected by the pulse wave detection unit 1 and amplitudes of the pulse wave that correspond to the reference times, and a pulse wave velocity detection unit 5 for finding a velocity of the pulse wave on basis of information representing the reference time and the amplitudes that is sent from the ejection and reflected waves information extraction unit 6.

The ejection and reflected waves information extraction unit 6 includes a reference time detection unit 2 for detecting the reference time for the identification of the ejection wave component included in the pulse wave detected by the pulse wave detection unit 1 and the reference time for identification of the reflected wave component included in the pulse wave.

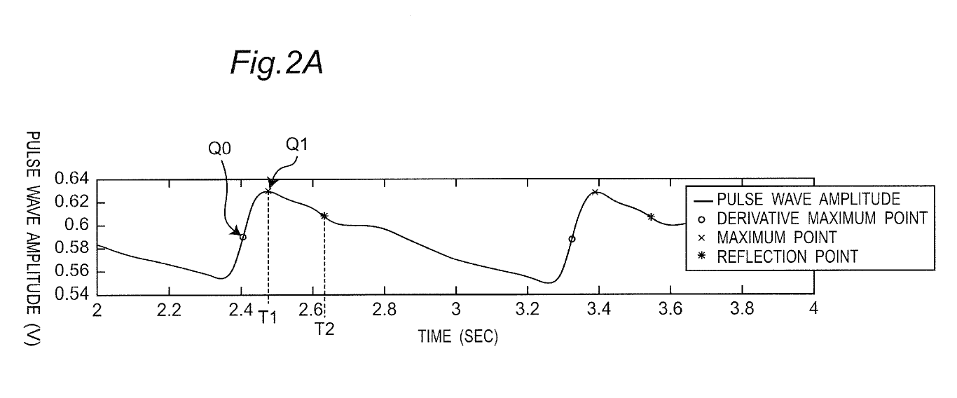

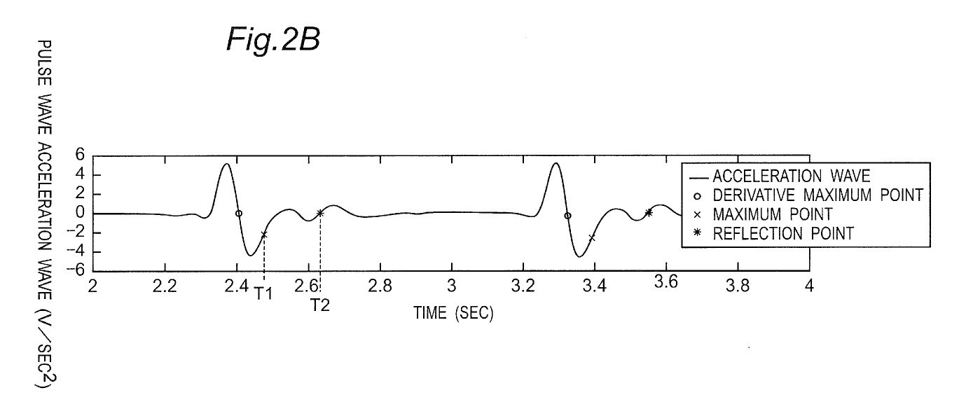

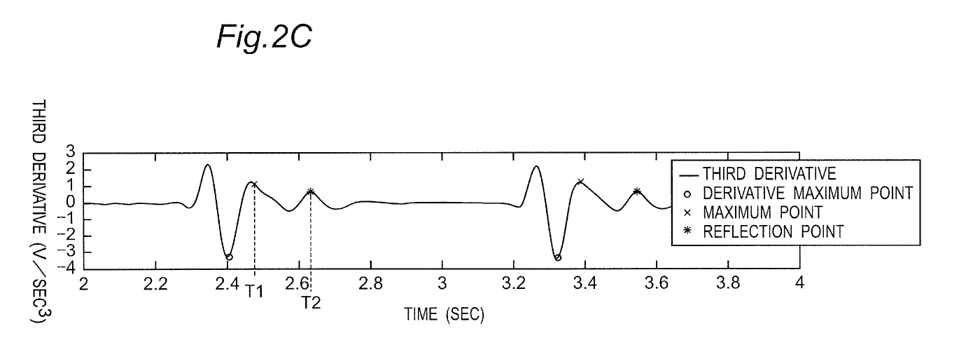

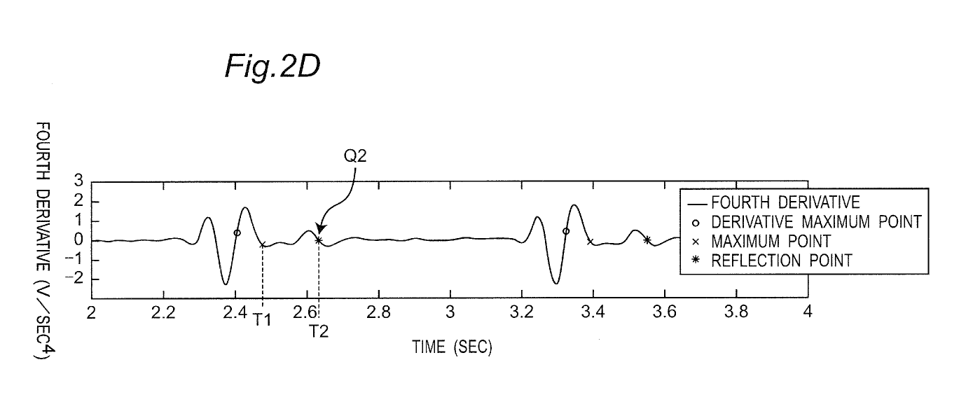

An example of a process in which the reference time detection unit 2 finds the reference times will be described below. FIG. 2A shows an example of a waveform of a pulse wave detected by the pulse wave detection unit 1. A vertical axis in FIG. 2A represents measured voltage values (V) corresponding to amplitudes (mmHg) of the pulse wave. In the pulse wave velocity measurement device 10 of the embodiment, correction (calibration) of correspondence of the voltage values (V) measured by the pulse wave detection unit 1 with the amplitudes (mmHg) of the pulse wave is performed on basis of blood pressures (mmHg) measured by a conventional cuff-type sphygmomanometer, as an example. The correction (calibration) has only to be performed when first use thereof is started, and a result of the calibration has only to be used for the subsequent measurement.

On condition that the pulse wave is of Type C in blood pressure waveform classification by Murgo et al., for instance, the reference time detection unit 2 detects time T1, at a maximum point Q1 in a systole in a waveform of the pulse wave shown in FIG. 2A, as reference time T1 for the ejection wave component. FIG. 2B shows an acceleration wave of the pulse wave, and FIG. 2C shows a third derivative of the pulse wave. On condition that the pulse wave is of Type C in the blood pressure waveform classification by Murgo et al., for instance, the reference time detection unit 2 detects a third zero cross point Q2 of a fourth derivative of the pulse wave shown in FIG. 2D, as reference time T2 for the reflected wave component. The third zero cross point Q2 signifies a point where the fourth derivative waveform shown in FIG. 2D crosses zero downward at the third time after the pulse wave shown in FIG. 2A passes the minimum value.

Though the above description has been given on the detected pulse wave that is of Type C in the blood pressure waveform classification, a method by which the reference time for the ejection wave component of the pulse wave and the reference time for the reflected wave component thereof can more preferably be determined may be employed, if any, on condition that the detected pulse waves have a waveform other than Type C in the blood pressure waveform classification. For instance, basically, pulse waves do not exhibit so great change in waveform unless there is great fluctuation in the blood pressure, and thus an accuracy in detecting a pulse wave can be improved by superimposition (arithmetic mean) of a plurality of measured pulse waves.

Pulse waves exhibit various waveforms according to noise levels, ages, sex, presence or absence of illness, physical conditions, and the like, and thus a method by which the reference time for the ejection wave component of the pulse waves and the reference time for the reflected wave component thereof can more preferably be determined may be employed. For instance, an accuracy in determining the reference time for the ejection wave component of the pulse wave and the reference time for the reflected wave component thereof can be improved by recording, as a history, of the detected pulse waves along with conditions of a measured person at that point of time.

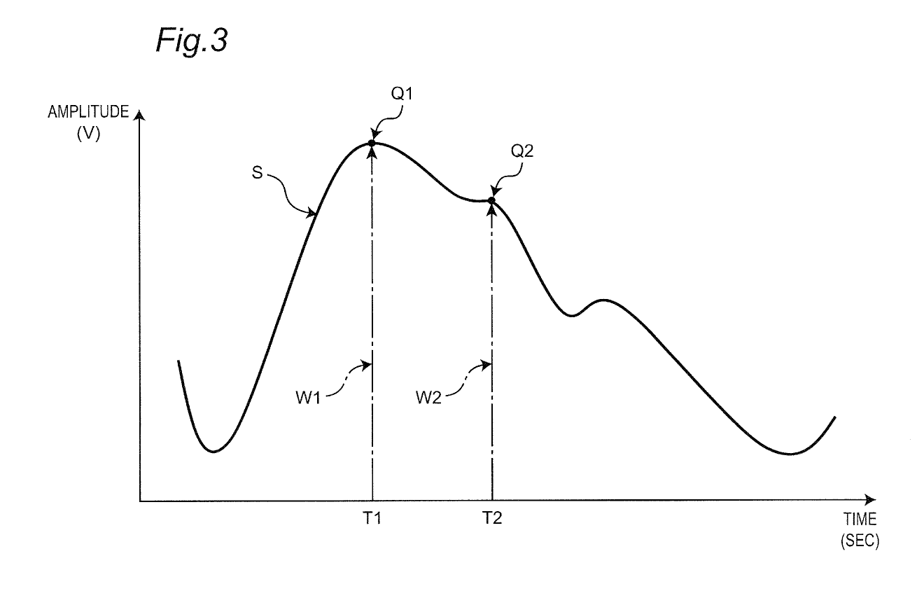

The ejection and reflected waves information extraction unit 6 includes a pulse wave amplitude detection unit 3. As shown in a waveform diagram of FIG. 3 as an example, the pulse wave amplitude detection unit 3 detects an amplitude W1 of the pulse wave S that corresponds to the reference time T1 for the ejection wave component detected by the reference time detection unit 2 and detects an amplitude W2 of the pulse wave S that corresponds to the reference time T2 for the reflected wave component detected by the reference time detection unit 2.

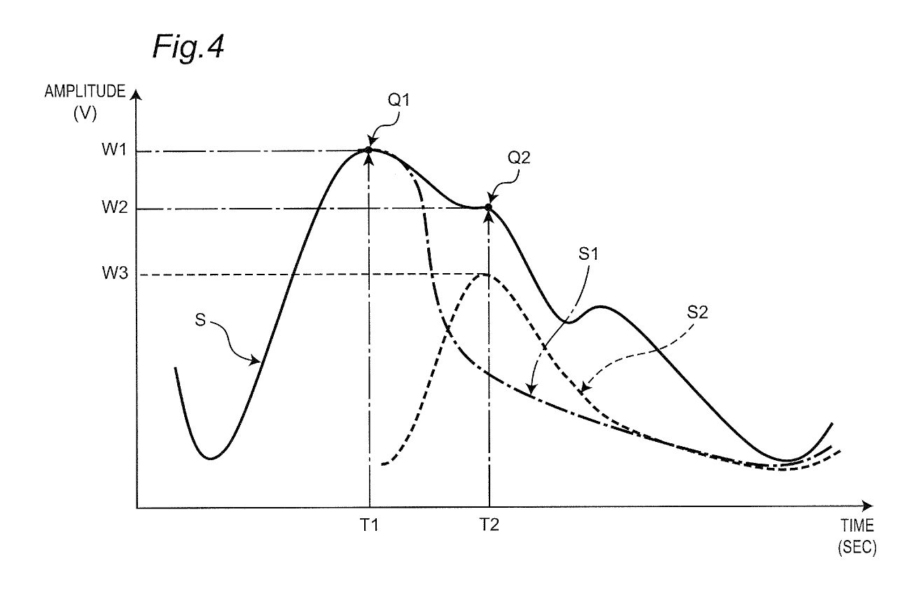

The ejection and reflected waves information extraction unit 6 further includes an ejection wave component elimination unit 4. As shown in FIG. 4, the ejection wave component elimination unit 4 eliminates an ejection wave component S1 of the pulse wave S from the amplitude W2 of the pulse wave S, which amplitude corresponds to the reference time T2 for the reflected wave component S2 and thereby finds an amplitude W3 of the reflected wave component S2, that corresponds to the reference time T2 for the reflected wave component S2. As a method of finding the amplitude W3 of the reflected wave component S2 that corresponds to the reference time T2 for the reflected wave component S2, a method is conceivable in which degrees of decrease in the ejection wave in the pulse wave are modeled with use of Windkessel model, for instance, and in which a remaining ejection wave component S1 is subtracted from the pulse wave amplitude measured around the reference time T2 for the reflected wave component S2. As a matter of course, a method by which the remaining ejection wave component S1 can more accurately be determined may be employed, if any.

The ejection and reflected waves information extraction unit 6 inputs into the pulse wave velocity detection unit 5 information representing the reference time T1 for the ejection wave component and the reference time T2 for the reflected wave component detected by the reference time detection unit 2 and inputs into the pulse wave velocity detection unit 5 information representing the amplitude W1 of the pulse wave S and the amplitude W3 of the reflected wave component S2 of the pulse wave S that have been detected by the pulse wave amplitude detection unit 3 and that correspond to the reference time T1 for the ejection wave component and the reference time T2 for the reflected wave components, respectively. Then the pulse wave velocity detection unit 5 finds the velocity of the pulse wave S on basis of the reference time T1 for the ejection wave component, the reference time T2 for the reflected wave component, the amplitude W1 of the pulse wave S that corresponds to the reference time T1 for the ejection wave component, and the amplitude W3 of the reflected wave component S2 of the pulse wave S that corresponds to the reference time T2 for the reflected wave component S2.

Subsequently, a process in which the pulse wave velocity detection unit 5 finds the pulse wave velocity PWV of the pulse wave S will be described.

For the pulse wave velocity, in general, pulse waves at two sites in a living body are measured and a velocity at which the ejection wave component of the pulse waves is propagated is found by a fundamental principle that is based on the principle of Moens-Korteweg. In Non-Patent Literature 2, a relation between the pulse wave velocity and the blood pressure is derived from the relation of Moens-Korteweg and is expressed as the following equation (1).

(PWV)²=α×exp(β×P) (1)

In the equation (1), PWV is the pulse wave velocity (m/sec), P is the blood pressure (mmHg), and a and β are constants that slightly change among individuals and according to measurement time even in an individual.

Application of the equation (1) to the pulse wave velocity PWV1 (m/sec) of the ejection wave S1 and the pulse wave velocity PWV2 (m/sec) of the reflected wave S2 provides the following equations (2) and (3), respectively.

(PWV1)²=α×exp(β×W1) (2)

(PWV2)²=α×exp(β×W3) (3)

In the equation (2), W1 is the pulse wave amplitude of the ejection wave S1, that is, the amplitude W1 of the pulse wave S that corresponds to the reference time T1 for the ejection wave S1 of FIG. 4, and the amplitude W1 corresponds to a pulse wave pressure of the ejection wave Sl. W3 therein is the pulse wave amplitude of the reflected wave S2, that is, the amplitude W3 of the reflected wave S2 that corresponds to the reference time T2 for the reflected wave S2 of FIG. 4, and the amplitude W3 corresponds to a pulse wave pressure of the reflected wave S2.

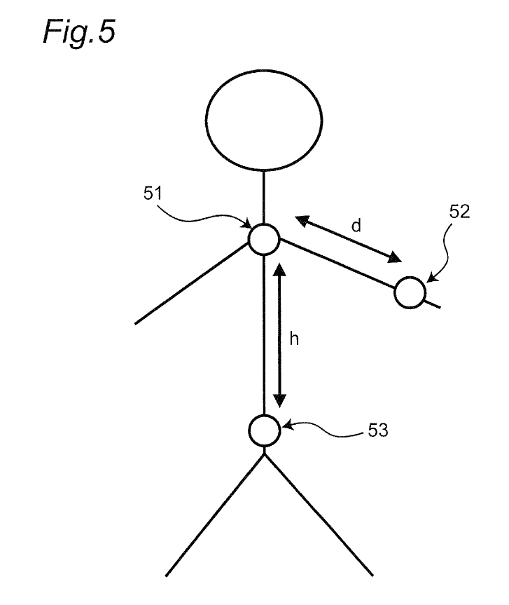

The following equations (4) and (5) are obtained with a distance from a heart 51 of a human body to a pulse wave measurement site 52 designated by d (m), a distance from the heart 51 to a reflection point 53 designated by h (m), a time period between a pulsation of the heart 51 and the reference time T1 for the ejection wave S1 designated by ΔT1 (sec), and a time difference (T2−T1) between the reference time T1 for the ejection wave S1 and the reference time T2 for the reflected wave S2 designated by ΔT2 (sec), as shown in FIG. 5.

(PWV1)²=(d/ΔT1)² (4)

(PWV2)²=((2h+d)/(ΔT1+ΔT2))² (5)

The following equation (6) is obtained from the equations (2) and (4) and the following equation (7) is obtained from the equations (3) and (5).

α×exp(β×W1)=(d/ΔT1)² (6)

α×exp(β×W3)=((2h+d)/(ΔT1+ΔT2))² (7)

The time ΔT1 can be eliminated from the equations (6) and (7). That is, the following equation (8) is obtained from the equation (7).

(ΔT1+ΔT2)²=(2h+d)²/α×exp(β×W3)

ΔT1+ΔT2=(2h+d)/{α×exp(β×W3)}^{1/2}

ΔT1=(2h+d)/{α×exp(β×W3)}^{1/2}−ΔT2 (8)

Substitution of the equation (8) for the equation (4) provides the following equation (9).

(PWV1)²=d²×[(2h+d)/α×exp{β×W3)}^{1/2}−ΔT2]⁻²

PWV1=d×[(2h+d)/α×exp(β×W3)]^{1/2}−ΔT2]⁻¹ (9)

That is, the pulse wave velocity PWV1 of the ejection wave S1 can be calculated from the known constants α, β, the distances d, h that are known measured values, the difference (T2−T1)=ΔT2 between the reference times T2 and T1 that are found by the reference time detection unit 2, and the amplitude W3 of the reflected wave S2, found by the ejection wave component elimination unit 4, that corresponds to the reference time T2 for the reflected wave S2.

Namely, the pulse wave velocity detection unit 5 finds the pulse wave velocity PWV1 of the ejection wave S1 of the pulse wave S, by the equation (9) derived from the equations (4) through (7) described above, on basis of the difference ΔT2 between the reference times T1 and T2 for the ejection wave component S1 and the reflected component S2, and the amplitudes W1, W3 of the ejection wave component S1 and the reflected component S2 of the pulse wave S. Thus the pulse wave velocity can be measured with high accuracy in consideration of a difference between the pulse wave velocities of the ejection wave and the reflected wave which difference is caused by a difference between the amplitude W3 of the ejection wave component S1 and the amplitude W3 of the reflected wave component S2.

The embodiment in which the pulse wave amplitude detection unit 3 includes the ejection wave component elimination unit 4 has been described above, whereas the following equation 10 in which the amplitude W3 of the reflected wave S2 that corresponds to the reference time T2 for the reflected wave S2 in the equation (3) is replaced by the amplitude W2 of the pulse wave S that corresponds to the reference time T2 for the reflected wave S2 is used providing that the pulse wave amplitude detection unit 3 does not include the ejection wave component elimination unit 4.

(PWV2)²=α×exp(β×W2) (10)

Under that condition, the following equation (11) is obtained from the equations (5) and (10).

α×exp(β×W2)=((2h+d)/(ΔT1+ΔT2))² (11)

Therefore, the following equation 12 is obtained by elimination of the time ΔT1 from the above described equation (6) and the equation (11).

ΔT1=(2h+d)/{α×exp(β×W2)}^{1/2}−ΔT2 (12)

Substitution of the equation (12) into the above described equation (4) provides the following equation (13).

(PWV1)²=d²×[{2h+d)/(α×exp(β×W2)}^{1/2}−ΔT2]⁻²

PWV1=d×[(2h+d)/α×exp(β×W2)]^{1/2}−ΔT2]⁻¹ (13)

That is, the pulse wave velocity PWV1 of the ejection wave S1 can be calculated from the known constants α, β, the distances d, h that are the known measured values, the difference (T2−T1)=ΔT2 between the reference times T2 and T1 that are found by the reference time detection unit 2, and the amplitude W2 of the pulse wave S that corresponds to the reference time T2 for the reflected wave S2 found by the pulse wave amplitude detection unit 3.

Namely, the pulse wave velocity detection unit 5 finds the pulse wave velocity PWV1 of the ejection wave S1 of the pulse wave S, by the equation (13) derived as described above, on basis of the difference ΔT2 between the reference times T1 and T2 for the ejection wave component Si and the reflected component S2, and the amplitude W2 of the pulse wave S at the reference time T2 that corresponds to the reference time T2 for the reflected wave component S2. Thus the pulse wave velocity can be measured with high accuracy in consideration of the difference between the pulse wave velocities of the ejection wave S1 and the reflected wave S2 of the pulse wave S.

Though the device for measuring the pulse wave velocity has been described for the embodiment, a blood pressure can be measured on basis of the pulse wave velocity measured in the embodiment, as apparent from the equation (1). That is, the pulse wave velocity found by the pulse wave velocity measurement device may be displayed as a blood pressure, instead of being displayed as it is, when a user uses the pulse wave velocity measurement device. That is because the blood pressure is a living-body index that is more familiar to people other than medical personnel than the pulse wave velocity though the pulse wave velocity is a living-body index that is extremely familiar to medical personnel.

A conventional sphygmomanometer using a cuff presents to a user a set composed of a systolic blood pressure, a diastolic blood pressure, a mean blood pressure, a pulse rate, and the like with use of several tens of seconds, whereas a sphygmomanometer based on the embodiment of the invention is capable of detecting a blood pressure for each pulsation and thus brings about a possibility that conditions of a living body can more minutely be grasped. Besides, the pulse wave velocity and the blood pressure can be detected thereby with higher accuracy by acquisition of an arithmetic mean, a moving average and/or the like of a unit of several pulsations.

By a pulse wave velocity measurement program, a computer may be made to execute a reference time deriving function of the reference time detection unit 2 finding the reference times T1 and T2, a pulse wave amplitude deriving function of the pulse wave amplitude detection unit 3 finding the amplitudes W1, W2 of the pulse wave S that correspond to the reference times T1, T2, an ejection wave component eliminating function of the ejection wave component elimination unit 4 finding the amplitude W3 of the reflected wave component S2 that corresponds to the reference time T2 for the reflected wave component S2, a function of the pulse wave velocity detection unit 5 finding the pulse wave velocity PWV1 of the ejection wave S1 of the pulse wave S on basis of the difference ΔT2 between the reference times T1 and T2 for the ejection wave component S1 and the reflected component S2 and the amplitudes W3 of the reflected component S2 of the pulse wave S that corresponds to the reference time T2 for the reflected wave component S2, as described above.

By a pulse wave velocity measurement program, the computer may be made to execute the reference time deriving function of the reference time detection unit 2 finding the reference times T1 and T2, the pulse wave amplitude deriving function of the pulse wave amplitude detection unit 3 finding the amplitudes W1, W2 of the pulse wave S that correspond to the reference times T1, T2, and a function of the pulse wave velocity detection unit 5 finding the pulse wave velocity PWV1 of the ejection wave S1 of the pulse wave S on basis of the difference ΔT2 between the reference times T1 and T2 for the ejection wave component S1 and the reflected component S2 and the amplitude W2 of the pulse wave S that corresponds to the reference time T2 for the reflected wave component S2, as described above.

### REFERENCE SIGNS LIST

1 pulse wave detection unit
2 reference time detection unit
3 pulse wave amplitude detection unit
4 ejection wave component elimination unit
5 pulse wave velocity detection unit 5
6 ejection and reflected waves information extraction unit
10 pulse wave velocity measurement device
Q1 maximum point
Q2 third zero cross point of fourth derivative
S pulse wave
S1 ejection wave
S2 reflected wave
T1 reference time for ejection wave component
T2 reference time for reflected wave component
W1 amplitude of pulse wave S that corresponds to reference time T1
W2 amplitude of pulse wave S that corresponds to reference time T2
W3 amplitude of reflected wave S2 that corresponds to reference time T2
PWV1 pulse wave velocity of ejection wave S1
PWV2 pulse wave velocity of reflected wave S2

1. A pulse wave velocity measurement device comprising:
a pulse wave detection unit (1) for detecting a pulse wave at one site in a living body,
a reference time detection unit (2) for detecting reference time for identification of an ejection wave component included in the pulse wave at the one site that is detected by the pulse wave detection unit (1) and reference time for identification of a reflected wave component included in the pulse wave,
a pulse wave amplitude detection unit (3) for detecting an amplitude of the pulse wave that corresponds to the reference time for the ejection wave component detected by the reference time detection unit (2) and detecting an amplitude of the pulse wave that corresponds to the reference time for the reflected wave component detected by the reference time detection unit,
a pulse wave velocity detection unit (5) for finding a velocity of the pulse wave at which the pulse wave propagates, on basis of the reference time for the ejection wave component and the reference time for the reflected wave component that have been detected by the reference time detection unit (2) and the amplitude of the pulse wave that corresponds to the reference time for the ejection wave component and the amplitude of the pulse wave that corresponds to the reference time for the reflected wave component which amplitudes have been detected by the pulse wave amplitude detection unit (3).

2. The pulse wave velocity measurement device as claimed in claim 1, wherein
the pulse wave amplitude detection unit (3) includes an ejection wave component elimination unit (4) for finding an ejection wave component included in the amplitude of the pulse wave that corresponds to the reference time for the reflected wave component, eliminating the ejection wave component from the amplitude of the pulse wave that corresponds to the reference time for the reflected wave component, and thereby finding an amplitude of the reflected wave component that corresponds to the reference time for the reflected wave component, and wherein
the pulse wave velocity detection unit (5) finds the velocity of the pulse wave on basis of the reference time for the ejection wave component and the reference time for the reflected wave component that have been detected by the reference time detection unit (2), the amplitude of the pulse wave that corresponds to the reference time for the ejection wave component that has been detected by the pulse wave amplitude detection unit (3), and the amplitude of the reflected wave component that has been found by the ejection wave component elimination unit (4).

3. The pulse wave velocity measurement device as claimed in claim 2, wherein
the pulse wave velocity detection unit (5) finds the velocity of the pulse wave on basis of a time difference between the reference time for the ejection wave component and the reference time for the reflected wave component that have been detected by the reference time detection unit (2), and an amplitude difference between the amplitude of the pulse wave that corresponds to the reference time for the ejection wave component and that has been detected by the pulse wave amplitude detection unit (3) and the amplitude of the reflected wave component that has been found by the ejection wave component elimination unit (4).

4. A pulse wave velocity measurement program that makes a computer execute:
a reference time deriving function of finding reference time for identification of an ejection wave component included in a pulse wave at one site in a living body and reference time for identification of a reflected wave component included in the pulse wave,
a pulse wave amplitude deriving function of finding an amplitude of the pulse wave that corresponds to the reference time for the ejection wave component and finding an amplitude of the pulse wave that corresponds to the reference time for the reflected wave component, and
a pulse wave velocity deriving function of calculating a velocity of the pulse wave at which the pulse wave propagates, on basis of the reference time for the ejection wave component, the reference time for the reflected wave component, the amplitude of the pulse wave that corresponds to the reference time for the ejection wave component, and the amplitude of the pulse wave that corresponds to the reference time for the reflected wave component.

5. The pulse wave velocity measurement program as claimed in claim 4, wherein
the pulse wave amplitude deriving function includes an ejection wave component eliminating function of finding an ejection wave component included in the amplitude of the pulse wave that corresponds to the reference time for the reflected wave component, eliminating the ejection wave component included in the amplitude of the pulse wave from the amplitude of the pulse wave that corresponds to the reference time for the reflected wave component, and thereby finding an amplitude of the reflected wave component that corresponds to the reference time for the reflected wave component, and wherein
the pulse wave velocity deriving function comprises calculating the velocity of the pulse wave on basis of the reference time for the ejection wave component, the reference time for the reflected wave component, the amplitude of the pulse wave that corresponds to the reference time for the ejection wave component, and the amplitude of the reflected wave component that has been found by the ejection wave component eliminating function.

6. The pulse wave velocity measurement program as claimed in claim 5, wherein
the pulse wave velocity deriving function comprises finding the velocity of the pulse wave on basis of a time difference between the reference time for the ejection wave component and the reference time for the reflected wave component, and an amplitude difference between the amplitude of the pulse wave that corresponds to the reference time for the ejection wave component and the amplitude of the reflected wave component that has been found by the ejection wave component eliminating function.
